(19) United States
(12) Patent Application Publication
(10) Pub. No.: US 2023/0075082 A1
(43) Pub. Date: Mar. 9, 2023

(54) METHOD FOR SYNTHESIZING DNA

(71) Applicant: SMOBIO Technology, Inc., Hsinchu City (TW)

(72) Inventor: Kuan-Lin Lee, Hsinchu City (TW)

(73) Assignee: SMOBIO Technology, Inc., Hsinchu City (TW)

(21) Appl. No.: 17/690,386

(22) Filed: Mar. 9, 2022

Related U.S. Application Data
(60) Provisional application No. 63/158,362, filed on Mar. 9, 2021.

(51) Int. Cl. C12P 19/34; C12N 15/10; C12N 9/00

(52) U.S. Cl. CPC C12P 19/34; C12N 15/1068; C12N 9/93

(57) ABSTRACT

The present invention provides a method for synthesizing DNA, which includes: (a) providing an oligonucleotide components library as a material for synthesizing DNA, wherein an oligonucleotide component is a short oligonucleotide chain whose new end and trailing end are OH groups; (b) analyzing sequence information of a DNA to be synthesized to obtain an oligonucleotide components combination order; (c) using the oligonucleotide components library to arrange an oligonucleotide component order according to the oligonucleotide components combination order; (d) phosphorylating the new end of a first-order oligonucleotide; (e) combining the first-order oligonucleotide with a complementary oligonucleotide of the first-order oligonucleotide to obtain a first-order double-stranded oligonucleotide; (f) combining a second-order oligonucleotide in the oligonucleotide components combination order with a complementary oligonucleotide of the second-order oligonucleotide to obtain a second-order double-stranded oligonucleotide; (g) ligating the phosphorylated new end of the first-order oligonucleotide with the trailing end of the second-order oligonucleotide to obtain a synthetic oligonucleotide having an OH group as the new end; and (h) repeating the above steps (d) to (g) to sequentially elongate the synthetic oligonucleotide until the DNA sequence to be synthesized is completed.

### CROSS-REFERENCE TO RELATED APPLICATION

This application claims the benefit of U.S. Provisional Patent Application No. 63/158,362, filed Mar. 9, 2021, which is incorporated by reference herein in its entirety.

### FIELD OF THE INVENTION

The present invention provides a novel method for synthesizing DNA.

### BACKGROUND OF THE INVENTION

For a long time, oligonucleotides have always been synthesized by way of organic chemistry. In 2019, the scientific community started a lively discussion on oligonucleotide synthesis by enzymatic reactions (Perkel, J. M. (2019). The race for enzymatic DNA synthesis heats up—An alternative to chemical oligonucleotide synthesis inches closer to reality, Nature 566, 565).

For enzymatic synthesis of oligonucleotides, the most important enzyme is Terminal Transferase TdT. It is an enzyme that is capable of adding one or more nucleotides to the OH group at the 3′ position of a double-stranded or single-stranded DNA. In general, uncontrolled TdT adds single nucleotides one by one, forming a long tail.

At present, there are two methods of enzymatic synthesis of DNA in the scientific field. One is used by ANSA Corporate, in which a single nucleotide is bound to the molecule of a single TdT enzyme. When DNA is synthesized by the enzyme TdT and nucleotides are added to the 3′ end of an oligonucleotide, the nucleotide (base) is added one at a time to avoid uncontrolled synthesis. Alternatively, each nucleotide added sequentially is chemically modified. The OH group at the 3′ end of the single nucleotide (dNTPs) is protected by a protecting group, and the OH group at the 3′ end is not exposed after being added to the 3′ end of the oligonucleotide. As a result, the nucleotide is not continuously reacted one after another. When elution is completed and ready for next reaction, the protection is then removed and the new OH group is exposed.

The single-reaction rate for current synthesis of oligonucleotides by organic chemistry is about 99%. However, when a longer long-chain oligonucleotide is to be synthesized, the error value is enlarged and the accuracy is greatly reduced. Therefore, it requires a time-saving, cost-effective, and highly accurate method for synthesizing DNA.

### BRIEF SUMMARY OF THE INVENTION

The present invention provides a method for synthesizing DNA, which comprises: (a) providing a oligonucleotide components library as a material for synthesizing DNA, wherein each oligonucleotide component is a short oligonucleotide chain whose new end and trailing end are OH groups; (b) analyzing the sequence information of a DNA to be synthesized to obtain an oligonucleotide components combination order; (c) using the oligonucleotide components library to arrange an oligonucleotide component order according to the oligonucleotide components combination order; (d) phosphorylating the new end of a first-order oligonucleotide; (e) combining the first-order oligonucleotide with a complementary oligonucleotide of the first-order oligonucleotide to obtain a first-order double-stranded oligonucleotide; (f) combining a second-order oligonucleotide in the oligonucleotide components combination order with a complementary oligonucleotide of the second-order oligonucleotide to obtain a second-order double-stranded oligonucleotide; (g) ligating the phosphorylated new end of the first-order oligonucleotide with the trailing end of the second-order oligonucleotide to obtain a synthetic oligonucleotide having an OH group as the new end; and (h) repeating the above steps (d) to (g) to sequentially elongate the synthetic oligonucleotide until the DNA sequence to be synthesized is completed.

### BRIEF DESCRIPTION OF THE DRAWINGS

FIG. 1 is a schematic diagram of the novel method for synthesizing DNA of the present invention.

FIG. 2 shows the first embodiment of the novel method for synthesizing DNA of the present invention.

FIG. 3 shows the second embodiment of the novel method for synthesizing DNA of the present invention.

FIG. 4 shows the third embodiment of the novel method for synthesizing DNA of the present invention.

FIG. 5 shows the fourth embodiment of the novel method for synthesizing DNA of the present invention.

FIG. 6 shows the fifth embodiment of the novel method for synthesizing DNA of the present invention.

### DETAILED DESCRIPTION OF THE INVENTION

In the following text, the preferred embodiments according to the present disclosure are described in details with reference to the drawings. The embodiments are exemplary, and are not intended as limitations on the scope of the invention. The following detailed description includes specific details to provide a thorough understanding of the embodiments of the present disclosure, but those skilled in the art may practice the present invention without these specific details.

As used herein, “a,” “an,” “the,” “at least one” and “one or more” are used interchangeably.

The present invention provides a novel method for synthesizing DNA, which can achieve a better effect by combining enzymatic reactions and chemical synthesis methods.

In DNA, there are a total of four bases, A, T, C and G, and an oligonucleotide is also composed of four different single nucleotides. For a short-chain oligonucleotide (short oligo), if it is composed of two bases, then the square of 4 results in 16 different oligonucleotides; if it is composed of three bases, the cube of 4 results in 64 different oligonucleotides; if it is composed of four bases, there are 256 different oligonucleotides; if it is composed of five bases, the exponent is 5, that is 1,024 different oligonucleotides; if it is composed of six bases, the exponent is 6, that is 4,096 different oligonucleotides.

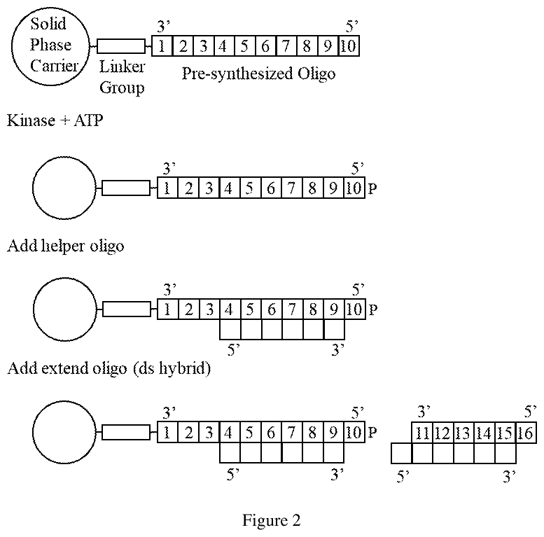

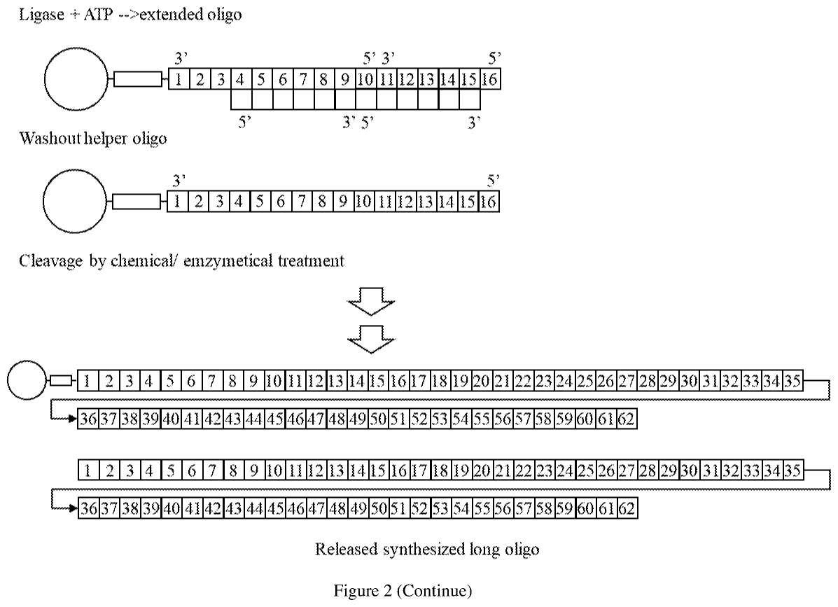

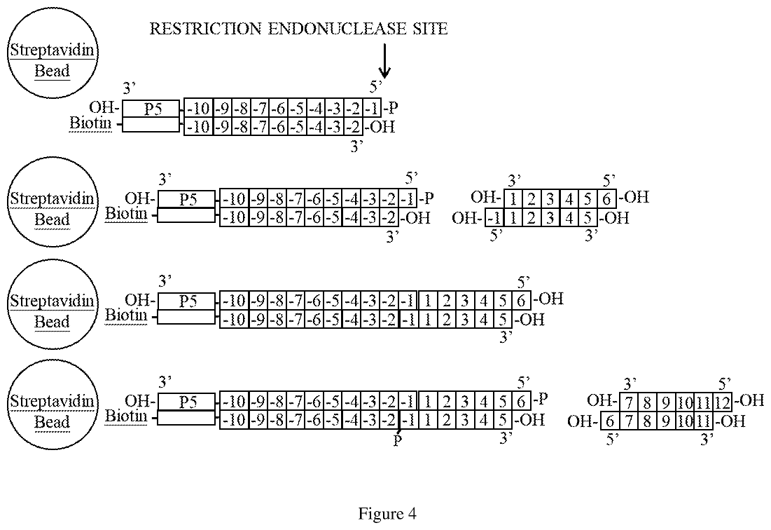

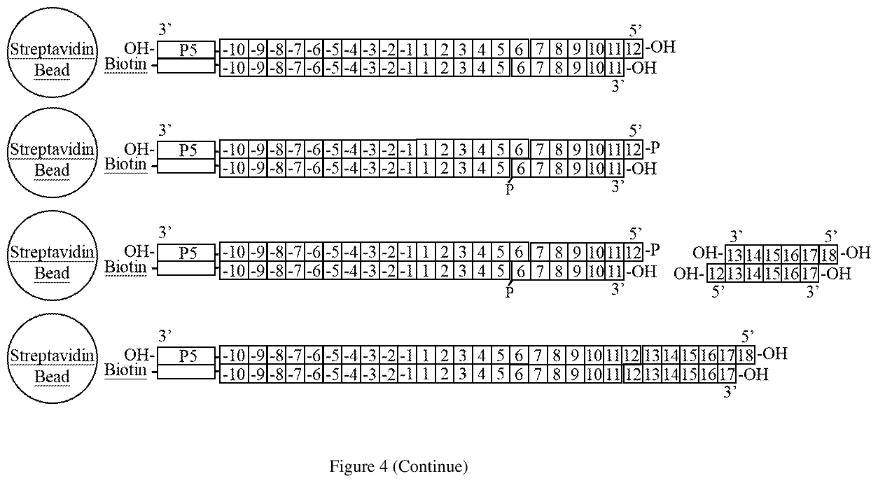

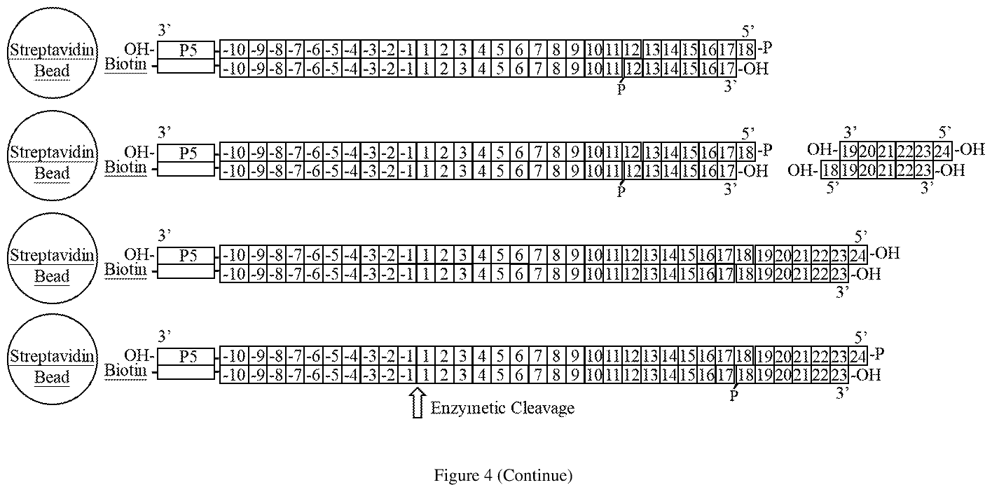

An oligonucleotide component library (short oligo library) is established first, which comprises a basic component group and a custom-made component group, wherein the length of the short oligo library is not limited to 5 to 8 oligonucleotides. A first-order oligonucleotide can be custom-made according to a synthesis scheme, and the length can be a single digit, or it can be longer than ten bases or tens of bases. The first oligonucleotide (Pre-synthesized Oligo) in FIG. 2 has 10 bases, and the first oligonucleotide (pre-synthesized oligo) in FIG. 4 further comprises a P5 sequence commonly used for PCR amplification, and the total length exceeds 30 bases.

The basic component group described in the present invention can be expanded to 4⁷=16,384 different oligonucleotides, and even 4⁸=65,536 different oligonucleotides. The advantage is that when the double strands are docked, the ligase has a larger range to hold and ligate, which is beneficial to the ligation reaction. Furthermore, synthesis of a full-length gene can be accomplished with fewer reactions. Moreover, by increasing the number of bases covered by the complementation of an oligonucleotide and a complementary oligonucleotide, the stability of the double-stranded oligonucleotide can be increased, especially so when the strength of hydrogen bonds between AT is lower than those between GC.

Besides, if oligonucleotides having the length of 7 or 8 bases are added to the short oligo library, the majority of synthetic elongation works can be implemented by using longer oligonucleotides having a length of 7 or 8 bases. Synthetic works other than the main synthetic work are then assembled from nucleotides having a length of 5 or 6 bases.

In addition to being used for synthesizing long-chain single-stranded or double-stranded DNAs, the present invention can also accomplish certain designs that are considered to be special to the art of conventional chemical syntheses of oligonucleotides, for example, I, U, or bases with special chemical modifications can be optionally added, for example, aminated, biotinylated, and single-stranded custom-made components are collectively form a “custom-made component group.” Double-stranded custom-made components assembled from oligonucleotides having complimentary sequences are provided through the “basic component group.”

The custom-made component group of the present invention refers to the following conditions: a nucleotide-oligonucleotide chain composed of nucleotides other than A, T, C, G, or an oligonucleotide short chain having a length of more than or equal to 10 nucleotides, or oligonucleotide sequence fragments that are not easy to be synthesized, such as oligonucleotide fragments containing same kinds of nucleotides with continuously repeated, for example, a poly A sequence synthesized from a DNA template of an artificial mRNA, or an oligonucleotide fragment having nucleotides that are the same and highly repetitive, for example, when a gene is artificially synthesized, the highly repetitive GC sequences in an eukaryotic promoter sequence.

The length of the custom-made components is not limited, it can be 5 to 8 bases in length similar to the basic component group, or it can be longer. The custom-made component probably needs to be custom-made because its length exceeds the scope of the component library.

In the process of implementing the present invention, the sequence information of a DNA to be synthesized is analyzed by a computer to obtain an oligonucleotide components combination order. The combination sequence is calculated by the computer to provide a plurality of possible suggestions, not just one; it is similar to stacking building blocks, a plurality of identical stacking results is formed, but they can be stacked with different shapes and different sizes of components in different order. For example, a DNA sequence to be synthesized contains 30 bases as follows:

ATGGCGCAGGTTCAGCTGGTTGAGTCTGGT

It can be synthesized by scheme (A) (8+6+8+8):

ATGGCGCA + GGTTCA + GCTGGTTG + AGTCTGGT.

It can be synthesized by scheme (B) (8+8+6+8):

ATGGCGCA + GGTTCAGC + TGGTTG + AGTCTGGT.

It can be synthesized by scheme (C) (12+6+6+6):

ATGGCGCAGGTT + CAGCTG + GTTGAG + TCTGGT.

If the synthesis is carried out according to scheme (A), though the ligation is carried out from the first-order to the fourth-order oligonucleotides in the order of 8+6+8+8 bases, there is a plurality of combinations when each complementary oligonucleotide is selected. The underlined ones are complementary oligonucleotides:

A1:
ATGGCGCA GGTTCA GCTGGTTG AGTCTGGT
TACCGCG TCCAAG TCGACCAA CTCAGACCA
A2:
ATGGCGCA GGTTCA GCTGGTTG AGTCTGGT
TACCGCG TCCAAGTC GACCAA CTCAGACCA
A3:
ATGGCGCA GGTTCA GCTGGTTG AGTCTGGT
TACCGCG TCCAAGT CGACCAA CTCAGACCA

In A1, it can be observed that when three ligations are made, they are all ligated with the 5′ protrude ends.

In A2, it can be observed that when three ligations are made, they are ligated with 5′ protrude end, 3′ protrude end, and 5′ protrude end.

In A3, it can be observed that when three ligations are made, they are respectively ligated with 5′ protrude end, blunt end, and 5′ protrude end.

When the present invention is implemented, if the protrude end is docked with the protrude end, as in the above-mentioned examples A1 to A3, the 5′ protrude ends are ligated with each other, and the 3′ protrude ends are ligated with each other, and the number of overhung bases at the protrude end is not necessarily only limited to one base, For example, the following example;

A4:
ATGGCGCA GGTTCA GCTGGTTGA GTCTGGT
TACCGC GTCCAAGT CGACCA ACTCAGACCA

In A3, it can be observed that when three ligations are made, they are respectively ligated with two overhung bases at 5′ protrude end, blunt end, three overhung bases at 5′ protrude end.

In the above example, the length of the complementary oligonucleotide of the fourth-order oligonucleotide in A1 is 9 bases, which exceeds the scope of the oligonucleotide basic component group. Certainly, under the condition that the synthesis target is a single-stranded DNA, only CTCAGACC is used to replace CTCAGACCA to avoid the trouble of preparing the custom-made components. The basic component mentioned here refers to oligonucleotides of which n is 8 (n=8), and there are 65,536 different oligonucleotides in total. When the present invention is implemented, the basic component group is expanded to N is 9 (N=9), there are 262,144 different oligonucleotides. It might be less economical and require higher management costs. When N is 10 (N=10), there are 1,048,576 different oligonucleotides, even higher management cost is required.

The first-order oligonucleotide atggcgcaggtt in scheme (C) is longer in length, and can also be regarded as a custom-made component; its complementary oligonucleotide can also be a custom-made component, and can also be selected from the basic components, and can be assembled complementarily. Although the length is not long enough to cover the whole process of the first-order oligonucleotide, it is acceptable so long as it is enough to form a structure required by enzymatic ligation.

When the present invention is implemented, “custom-made components” can be used to produce the fragments of the regions that are difficult to be synthesized, for example, a DNA sequence to be synthesized that contains 300 bases, in which there are two regions. The base site of A region is 31-60, and the sequence is GGGGGCCGGGGCCGGGGGGGCCGGGGGCCG. It is a segment that is rich in GC and a highly repetitive region. When analyzing the sequence, it indicates that the basic components are difficult to be assembled, then the fragments of the regions can be synthesized with custom-made components. The base site of B region is 271-300, and the sequence is 30 consecutive A (AAAAAAAAAA AAAAAAAAAA AAAAAAAAAA). It can also be synthesized with custom-made components. The number of bases for synthesizing each order of the oligonucleotides can be arranged as 8+6+8+8+30+8+6+8+8+8+6+8+8+6+6+6+6+6+8+6+8+8+8+6+8+8++6+6+6+6+6+8+6+8+8+30, in which the first 30 (the fifth-order nucleotide) and the second 30 (the 36th-order nucleotide) are respectively two custom-made components for being used to synthesize the A region, the base site is 31-60, and the B region, the base site is 271-300. The complementary oligonucleotides of the two custom-made components can be custom-made or selected from the basic components for being assembled complementarily. Although the length is not enough to cover the whole process of the custom-made oligonucleotides, it is acceptable so long as it is enough to form the end structure required for enzymatic ligation.

The A region, the sequence site is 31-60, can be flexibly designed when custom-made components are prepared, for example, When the sequence whose sequence site is 23-68 is the fourth-order oligonucleotide, the number of bases for synthesizing each order of oligonucleotides can be arranged as 8+6+8+46+6+8+8+8+6+8+8+6+6+6+6+6+8+6+8+8+8+6+8+8++6+6+6+6+6+8+6+8+8+30. The advantage of this arrangement is that in a highly repetitive sequence region, for example from 31 to 60, even if a custom-made complementary oligonucleotide is synthesized, there is a good chance that a double-stranded oligonucleotide having accurate complementary base pairing is difficult to be formed. When the front and back, the upstream and downstream contain sequence regions that are not high in GC and not highly repetitive, a double-stranded oligonucleotide with accurate complementary base pairing is expected to be effectively formed, which is beneficial to the synthesis work. For example, when the base site is 31-60, the sequence is GGGGGCCGGGGCCGGGGGGGCCGGGGGCCG and forms the following structure with a complementary oligonucleotide:

GGGGGCCGGGGCCGGGGGGGCCGGGGGCCG
ACCCCCGGCCCCGGCCCCCCCGGCCCCCGG

Such a structure is prone to dislocation.

When the base site is changed to 23-68, and the sequence is AGTCTGGTGGGGGCCGGGGCCGGGGGGGCCGGGGGCCGAATC TACA, it is easier to be assembled with the complementary oligonucleotide to form the following structure:

AGTCTGGTGGGGGCCGGGGCCGGGGGGGCCGGGGGCCGAATCTACA
CTCAGACCACCCCCGGCCCCGGCCCCCCCCGGCCCCCGGCTTAGATG

The present invention provides a method for synthesizing DNA, which comprises: (a) providing an oligonucleotide components library as a material for synthesizing DNA, wherein each oligonucleotide component is a short oligonucleotide chain whose new end and trailing end are OH groups; (b) analyzing the sequence information of a DNA to be synthesized to obtain an oligonucleotide components combination order; (c) using the oligonucleotide components library to arrange an oligonucleotide component order according to the oligonucleotide components combination order; (d) phosphorylating the new end of a first-order oligonucleotide; (e) combining the first-order oligonucleotide with a complementary oligonucleotide of the first-order oligonucleotide to obtain a first-order double-stranded oligonucleotide; (f) combining a second-order oligonucleotide in the oligonucleotide components combination order with a complementary oligonucleotide of the second-order oligonucleotide to obtain a second-order double-stranded oligonucleotide; (g) ligating the phosphorylated new end of the first-order oligonucleotide with the trailing end of the second-order oligonucleotide to obtain a synthetic oligonucleotide having an OH group as the new end; and (h) repeating the above steps (d) to (g) to sequentially elongate the synthetic oligonucleotide until the DNA sequence to be synthesized is completed.

In one embodiment, the present invention discloses a novel method for synthesizing DNA, wherein the first-order oligonucleotide further comprises a nucleotide sequence for enzymatic recognition and cleavage.

In one embodiment, the present invention discloses a novel method for synthesizing DNA, wherein the order of step (d) and step (e) is interchangeable.

In another embodiment, the present invention discloses a novel method for synthesizing DNA, which further comprises the following step after performing step (c) and before performing step (f): combining 3′ end located at the first-order oligonucleotide sequence with a solid-state matrix through a linker group, wherein the linker group is cleavable.

In one embodiment, the linker group is cleaved by heating, changing pH values, redox reaction or enzymatic decomposition.

In one embodiment, the linker group is a small fragment of oligonucleotide with linking function, one end is mainly and directly bound with the solid-state matrix, the other end is ligated with the first-order oligonucleotide component to be synthesized, and the linker group has a segment of sequence capable of being recognized and cleaved by enzymes.

In another embodiment, not only the oligonucleotide linker group itself can be combined with the solid-phase matrix, its sequence is complementary to the first-order oligonucleotide, so that the first-order oligonucleotide is indirectly fixed onto the solid-phase matrix solely by hydrogen bonds. The first-order oligonucleotide comprises the starting point of the “DNA sequence to be synthesized.” It further comprises additional sequences required for providing complementary functions and enzymatic cleavage and recognition. The first oligonucleotide is often a custom-made component due to its long length, and its synthesis needs to be customized.

In one embodiment, if the length of the finally synthesized first-order oligonucleotide of the present invention is long enough, after the entire synthesis scheme is completed, an appropriate amount of complementary oligonucleotide components can be added. The added complementary oligonucleotide components and the first oligonucleotide form a complementary double strands for enzymatic recognition, or an enzymatic cleavage site is designed in the first oligonucleotide, so that the newly synthesized long-chain oligonucleotide can be cleaved with enzymes.

In one embodiment, a step is further included after step (h): eluting the complementary strand of oligonucleotide sequence of the synthesized oligonucleotide to obtain a synthetic single-stranded DNA; in another embodiment, the complementary strand of oligonucleotide sequence of the synthesized oligonucleotide is eluted by using physical or chemical actions; in a preferred embodiment, the complementary strand of oligonucleotide sequence of the synthesized oligonucleotide is eluted by heating or changing pH values.

In one embodiment, the new end (5′ end) of the first-order oligonucleotide sequence in step (g) is ligated with the trailing end (3′ end) of the second-order oligonucleotide sequence through an enzyme, and the enzyme is a double stranded DNA ligase.

The present invention is a novel method for synthesizing DNA, wherein the ligating method used by a linking molecule comprises: avidin (or streptavidin) biotin interactions, covalent immobilization or click chemistry, wherein the covalent immobilization is a disulfide bonding.

In one embodiment, the length of the short oligonucleotide chain of the oligonucleotide component is n and n-1 bases, and n is an integer greater than or equal to 2; in a preferred embodiment, n is 6-8; in a more preferred embodiment, n is 6, and this embodiment is an oligonucleotide component of the basic component group, and it does not include the custom-made component group.

In one embodiment, the length of the short oligonucleotide chain of the oligonucleotide component is n and n-2 bases, and n is an integer greater than or equal to 3; in a preferred embodiment, n is from 6 to 8; in a more preferred embodiment, n is 6, and this embodiment is an oligonucleotide component of the basic component group, and it does not include the custom-made component group.

In one embodiment, the length of the short oligonucleotide chain of the oligonucleotide component is n and n-3 bases, and n is an integer greater than or equal to 4; in a preferred embodiment, n is from 6 to 8; in a more preferred embodiment, n is 6, and this embodiment is an oligonucleotide component of the basic component group, and it does not include the custom-made component group.

In one embodiment, the length of the short oligonucleotide chain of the oligonucleotide component is n and n-4 bases, and n is an integer greater than or equal to 5; in a preferred embodiment, n is from 6 to 8; in a more preferred embodiment, n is 6, and this embodiment is an oligonucleotide component of the basic component group, and it does not include the custom-made component group.

In one embodiment, the length of the short oligonucleotide chain of the oligonucleotide component is n and n-5 bases, and n is an integer greater than or equal to 6; in a preferred embodiment, n is from 6 to 8; in a more preferred embodiment, n is 6, and this embodiment is an oligonucleotide component of the basic component group, and it does not include the custom-made component group.

The “new end” (Novo) described herein refers to 5′ end in the nucleotide sequence; and the “trailing end” refers to 3′ end in the nucleotide sequence.

After the new end (5′ end) of the first-order nucleotide sequence is phosphorylated by T4 polynucleotide kinase (T4 DNA ligase), the ability of ligating the next nucleotide sequence is acquired. Therefore, the trailing end (3′ end) of the second-order nucleotide sequence to be added subsequently is successfully ligated and will not be cleaved by subsequent steps such as heating or changing pH values, thereby maintaining the stability of the synthesized DNA. As a result, the 5′ end of the single-strand of the synthesized DNA is an un-phosphorylated new end.

The term “double-stranded DNA” referred herein includes a complete double-stranded DNA, a single-stranded DNA having a nick or a double-stranded DNA having a partially stripped single strand. The synthesis process is mainly a phosphorylation process of only the 5′ end of the first-order oligonucleotide component to form the so-called activated new end. During the synthesis, the 3′ end of the first-order complimentary oligonucleotide component is provided only with OH group. In each synthesis, the 5′ end of the second-order complementary oligonucleotide is not phosphorylated. As a result, there is no actual ligation between them, forming a phenomenon of nick. The single-stranded complementary oligonucleotide is easy to fall off, even so during the synthesis process.

### EMBODIMENTS

The present invention relates to a method for synthesizing DNA, and its specific implementation is a synthesis from 3′ end to 5′ end.

The following examples are not intended to be limiting, and are used solely for presenting various aspects of the present invention. Each block represents a nucleotide, and the code thereon is used to represent the nth nucleotide. If n<0, it means that it is the last nucleotide to be removed enzymatically or chemically.

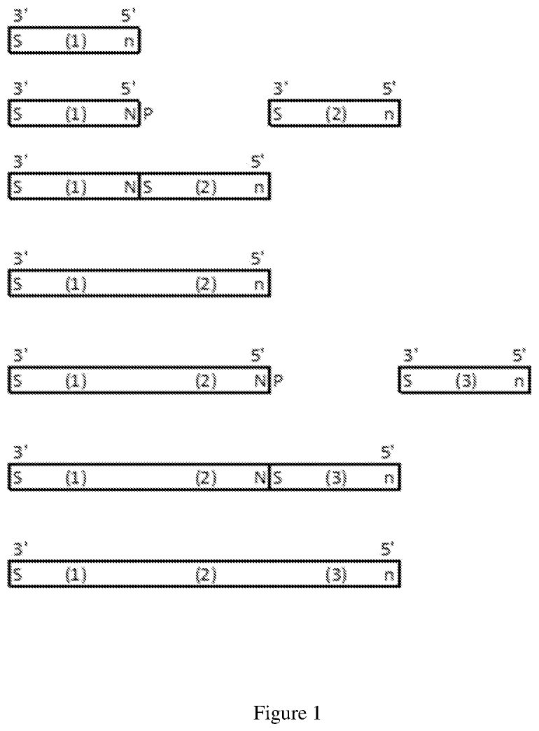

The main synthesis method of the present invention is shown in FIG. 1. Taking two fragments of oligonucleotide sequence as an example, the two ends of the first-order oligonucleotide sequence were the trailing ends (3′ end) and the new end (5′ end), respectively. At this time, both ends were not provided with the ability to ligate with other molecules or nucleotide sequences. Through phosphorylation of the new end, wherein the n-terminus was the new end (5′ end) that was not phosphorylated, and the N-terminus was the new end (5′ end) that was phosphorylated. Therefore, the reactions were proceeded in sequence from n to N, so that the first-order oligonucleotide sequence was able to ligate with other nucleotide sequences. At this time, the trailing end of the second-order oligonucleotide sequence was able to ligate with the phosphorylated new end of the first-order oligonucleotide sequence to form a new first-order nucleotide sequence, and both ends were new ends or trailing ends having no ability to ligate.

As shown in FIG. 1, FIG. 1 only briefly shows the synthesis process of the sequence to be synthesized, in which the N-terminus and S-terminus were connected by magnets in series as an example to illustrate the present invention.

In one embodiment, short oligonucleotides having five bases or six bases in length were designed by the present invention, resulting in a total of (1024+4096)=5120 different short oligonucleotides. Since almost any length could be assembled from fragments having five and six bases in length, therefore, oligonucleotides as short as 10 bases (10 mers) and as long as hundreds of bases could be assembled from fragments having five and six bases by synthesis from 3′ end to 5′ end.

The present invention provided a method for synthesizing DNA, which comprises: (a) providing an oligonucleotide components library as a material for synthesizing DNA, wherein each oligonucleotide component is a short oligonucleotide chain whose new end and trailing end are OH groups; (b) analyzing the sequence information of a DNA to be synthesized to obtain an oligonucleotide components combination order; (c) using the oligonucleotide components library to arrange an oligonucleotide component order according to the oligonucleotide components combination order; (d) phosphorylating the new end of a first-order oligonucleotide; (e) combining the first-order oligonucleotide with a complementary oligonucleotide of the first-order oligonucleotide to obtain a first-order double-stranded oligonucleotide; (f) combining a second-order oligonucleotide in the oligonucleotide components combination order with a complementary oligonucleotide of the second-order oligonucleotide to obtain a second-order double-stranded oligonucleotide; (g) ligating the phosphorylated new end of the first-order oligonucleotide with the trailing end of the second-order oligonucleotide to obtain a synthetic oligonucleotide having an OH group as the new end; and (h) repeating the above steps (d) to (g) to sequentially elongate the synthetic oligonucleotide until the DNA sequence to be synthesized is completed.

The novel method for synthesizing DNA of the present invention further comprised the following step after performing step (c) and before performing step (f): ligating the 3′ end located at the first-order oligonucleotide sequence with a solid-phase matrix through a linker group, wherein the linker group was cleavable.

The present invention is a novel method for synthesizing DNA, which further comprised a step (i): eluting the complementary strand of oligonucleotide sequence of the synthetic oligonucleotide to obtain a synthetic single-stranded DNA.

### Example 1

As shown in FIG. 2, a single nucleotide/short oligonucleotide copolymer was established from a solid-phase origin starting from the 3′ end. The surfaces of these solid-phase matrixes were ligated with cleavable linker group (Linker) which were cleaved after all of the reactions were completed to release the synthesized long oligonucleotide sequences, i.e., the synthetic DNA.

As shown in FIG. 2, steps (d)-(g) were repeated to determine the oligonucleotide sequence to be synthesized, and then analysis was conducted to obtain the oligonucleotide component to be added. In addition, nucleotide kinase (polynucleotide kinase) was used to phosphorylate the new end (5′ end) of the first-order oligonucleotide sequence to form an activated new end. Then the first-order oligonucleotide sequence was ligated with a complementary oligonucleotide sequence to obtain a 5′ or 3′ overhung double strand. The second-order oligonucleotide located in the oligonucleotide components combination order was combined with a complementary oligonucleotide to obtain a double strand having a second-order oligonucleotide overhung 3′ end or a complementary oligonucleotide sequence overhung 5′ end. The other ends of the double strand could be protrude ends or blunt ends, and then the 3′ ends of the double-stranded oligonucleotide sequence was ligated with the 5′ end of the first-order oligonucleotide sequence using a double stranded DNA ligase to obtain a synthetic oligonucleotide with an OH group at the 5′ end. Then the complementary strand of oligonucleotide sequence of the synthesized oligonucleotide was eluted by using physical or chemical actions, the step was repeated to elongate the oligonucleotide sequence on the solid-phase matrix by adding oligonucleotides again and again, and the linker was finally cleaved to obtain the final oligonucleotide product.

### Example 2

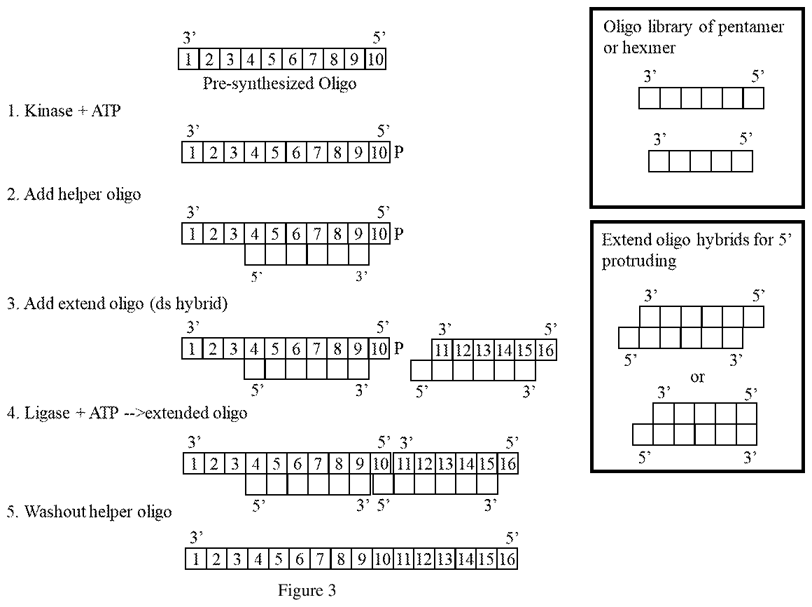

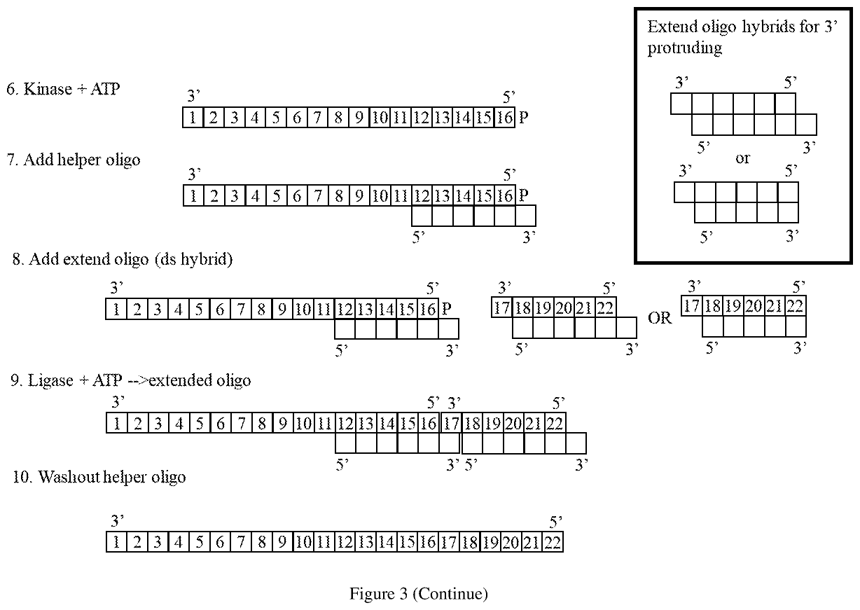

Similar to steps (a) to (h) of the synthesis process described above, when the DNA sequence was elongated in the present invention, the second-order oligonucleotide sequence itself was not only arranged according to the originally determined nucleotide order, the overhang at the new end was further modified according to the first-order double-stranded oligonucleotide sequence to be ligated. As shown in FIG. 3, the modification methods included setting the number of nucleotides as a group of five or six, setting the double-stranded ligated part in the second-order oligonucleotide sequence that was mainly ligated with the first-order oligonucleotide as the 3′ end overhung or the 5′ end overhung, and optionally setting the other end as single-stranded protrude ends or blunt ends as required. Since only the 5′ end of the new end in the first-order was phosphorylated, when the oligonucleotide next in line formed two strands, the trailing end was not phosphorylated. As a result, even if the blunt end and the blunt end were docked, neither two strands flipping nor reverse ligation at the tailing end could happen.

In addition, after the synthesis was completed, because only the 5′ end of one strand was phosphorylated during the ligation process, and the OH group was maintained at the 5′ end of the other strand, even if several second-order nucleotide complementary sequences were subsequently ligated, the complementary sequence of the synthesized DNA sequence was a sequence in a nick condition. There was no tight connection between them, which facilitated subsequent physical or chemical elution so that the complementary sequence could be eluted more quickly to leave a single-stranded DNA synthesis sequence.

### Example 3

Basically, steps (d) to (g) were repeated as described above, which was characterized in that, after the synthesis was completed, an oligonucleotide sequence complementary to the first-order oligonucleotide sequence could be further added as a primer or an oligonucleotide sequence complementary to the upstream sequence of the first-order oligonucleotide sequence could be included as a primer, and then reacted with DNA polymerase to obtain a complete double-stranded DNA product. As the example shown in FIG. 4, the used P5 sequence was the well-known P5 primer sequence commonly used in NGS sample amplification and preparation, which had the function of allowing DNA polymerase to undertake PCR reactions after the synthesis was completed. At the same time, the first-order oligonucleotide was ligated with the complementary oligonucleotide solely by hydrogen bonds to be indirectly immobilized onto the solid-phase matrix.

### Example 4

The embodiment as shown in FIG. 4, similar to the synthesis steps from (a) to (h) described above, compared to the process described in Example 1, the difference lies in the linker group (LG), biotin in this example, it was initially ligated with the 5′ end of the complementary sequence of the first-order double-stranded nucleotide sequence. During the first elongation of the DNA sequence, as before, only the 5′ end of the single strand (also referred to as the new end in the present invention) was phosphorylated, so that it was able to be ligated with the trailing end (3′ end) of the second-order double-stranded nucleotide sequence. However, when the second elongation was undertaken, in addition to the 5′ end of the DNA originally to be synthesized was phosphorylated, the 5′ end, if a nick was found, of the complementary strand was also phosphorylated, which in turn was ligated and repaired by ligase. As a result, when the synthesis was finally completed, a nearly complete double-stranded DNA was formed.

### Example 5

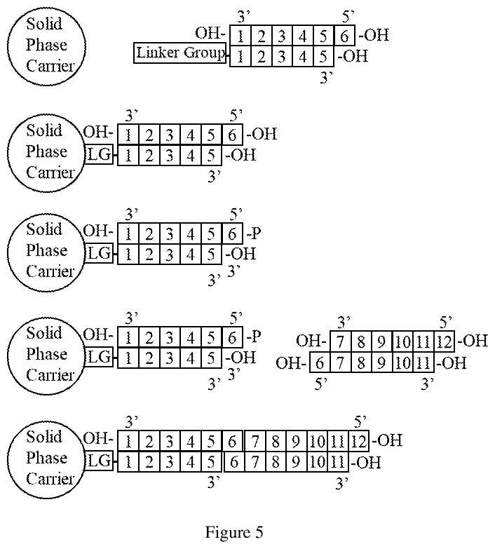

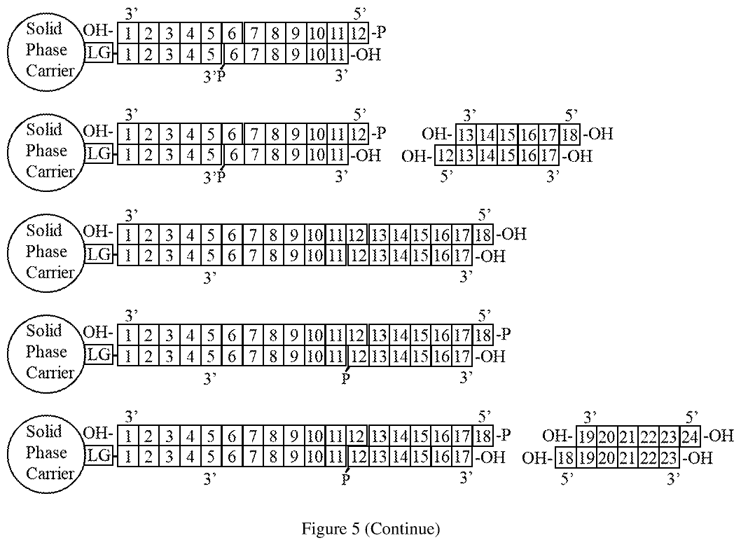

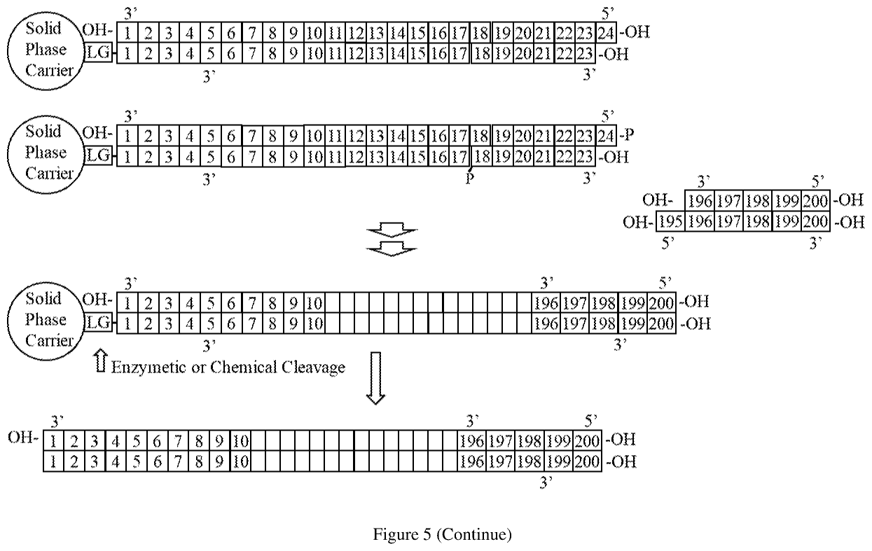

As shown in FIG. 5, one end of the linker group LU was bound with the solid-phase matrix, and the other end was ligated with the complementary oligonucleotide, and the first-order oligonucleotide was indirectly fixed to the solid-phase matrix by means of hydrogen bonding. The linker group could certainly be designed directly at the 3′ end of the first-order oligonucleotide, so that the first-order oligonucleotide was directly immobilized on the solid-phase matrix.

The first-order oligonucleotide, one end of the start oligonucleotide was joined with the linker group (LG) and the solid-phase matrix, and the other end was responsible for performing steps (d)-(g), after proceeding the step of finally completing DNA synthesis, the completely synthesized oligonucleotide could be stripped off from the solid-phase matrix by using a method capable of cleaving the linker group. If the linker group had a disulfide bond (—SS—), it was cleaved (—SHHS—) with a reducing agent. If the linker group was a polypeptide, it was cleaved with a suitable protease, for example, the LVPRGS sequence was cleaved with Thrombin. The disulfide bonds should be immobilized onto the solid-phase matrix in a manner of covalent bonding, and the immobilization method could be the common organic chemical reactions, for example, proceeded with click chemistry.

The nicks between the complementary oligonucleotides as shown in FIG. 5 were phosphorylated altogether in repeated phosphorylation processes, and were ligated by subsequent actions of the ligase. The actions were commonly seen before the PCR reaction, it was similar to performing the repair of DNA sample template. Though this reaction is not essential to the present invention, since it is shown in the drawing, the description is hereby supplemented.

### Example 6

One end of the linker group was bound to the solid-phase matrix, and the other end was connected to the complementary oligonucleotide, and the first-order oligonucleotide was indirectly immobilized onto the solid-phase matrix by means of hydrogen bonding. The linker group could certainly be designed directly on the 3′ end of the first-order oligonucleotide, so that the first-order oligonucleotide was directly immobilized on the solid-phase matrix.

When an enzymatic cleavage site was added to the upstream of the first-order oligonucleotide sequence, when the DNA sequence to be synthesized reached the target length, the long-chain oligonucleotide could be cleaved with an enzyme.

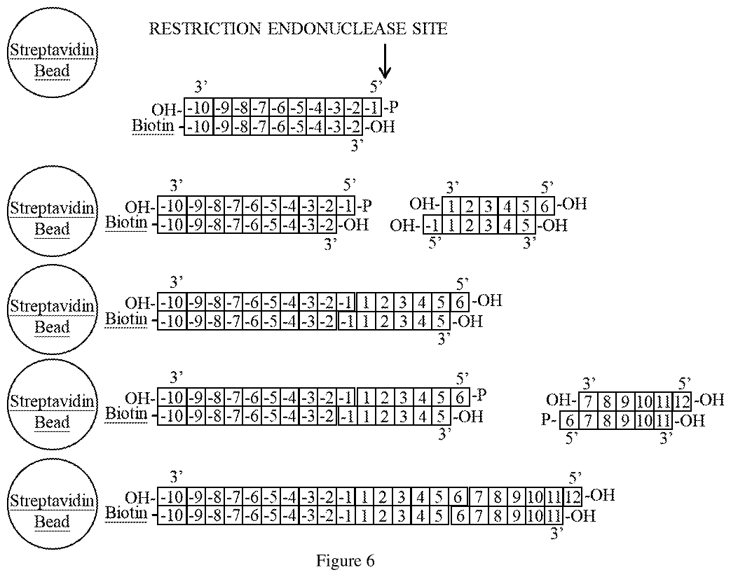

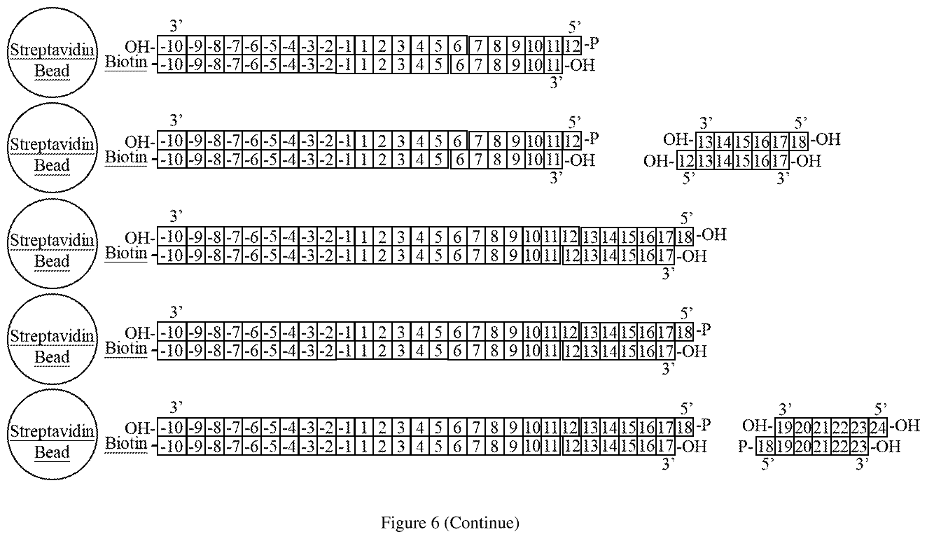

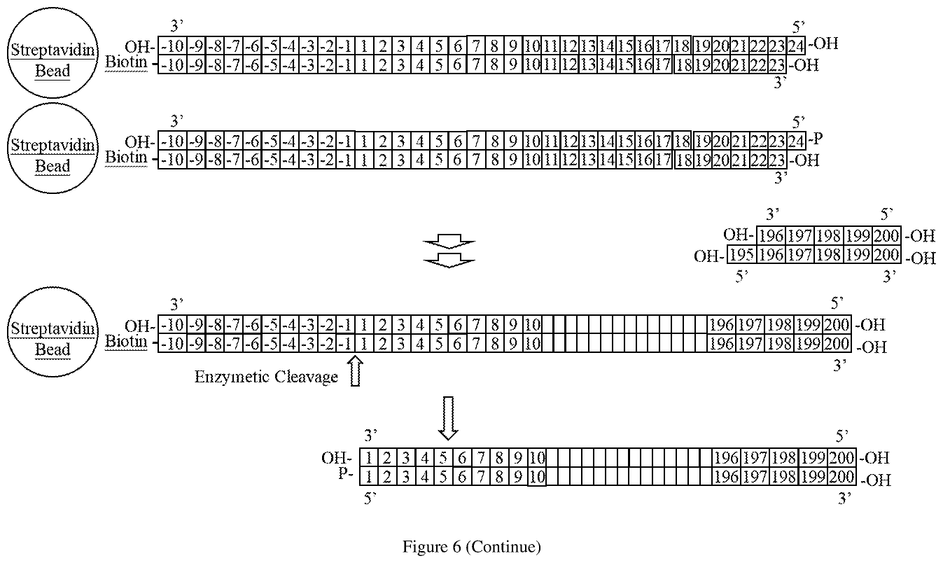

As shown in FIG. 6, the most significant feature was that the start sequence was not the DNA sequence to be synthesized, instead, it was a sequence that was capable of being cut by an enzyme. The enzyme was a restriction enzyme or a nickase, wherein one end was bound to the linker group (biotin) and the solid-phase matrix, the other end was responsible for performing steps (d)-(g). The biggest difference from previous embodiments was that the nucleotides of the DNA sequence to be synthesized were counted from the first ligation of the 2-position nucleotide sequence, and after proceeding to the final DNA synthesis step, enzymes could be used to directly remove the remaining start sequence, the linker group and the solid-phase matrix.

Enzymes was used as a means of final cleavage, and at least one nucleotide was reserved for being cleaved by enzymes. Taking this embodiment as an example, only the new end of the first-order oligonucleotide was required to be designed as a protrude end.

### Example 7

The present invention also had environmental friendly features. For example, when the synthesis target was 70 nucleotides (the chain was often 70-mer), and the number of nucleotides to be ligated each time was 7 short nucleotide chains (the chain length was 7-mer), according to the current DNA synthesis method, there were chemical method and enzyme method used in the present invention. Assuming that the chemical reaction rate was 99% and the enzyme reaction rate was 95%, according to the results of the chemical method, a total of 69 reactions were required, and the final successful yield was (99%)∧69=49.98%, and 50.02% of the raw material was discarded after further purification.

The enzyme method of the present invention was divided into a single synthesis of 7 nucleotides, which included 6 chemical ligation processes, the yield rate was (99%)∧6=94.15%, after being purified, 5.85% of the raw material was discarded. In addition, 10 short nucleotide sequences were subjected to enzyme reaction for 9 times, and the yield was (95%)∧9=63%, after purification, 37% was discarded. The raw material totally discarded was 37%+5.85%=42.85%. It was apparently fewer than the raw material discarded by pure chemical synthesis.

After the synthesis was completed, the finished product needed to be further purified by using HPLC. Based on the results of the chemical method, the impurity level was 1, in other words, except for the nucleotides having a chain length of 70-mer as the purification target, all nucleotides having a chain length of 69-mer, 68-mer . . . 1-mer were required to be moved, rendering difficult separations and purifications.

On the contrary, after the DNA sequence was synthesized by the enzyme method of the present invention, the impurity level was 7-mer, in other words, the targets to be analyzed were those having a chain length of 70-mer, 63-mer, 56-mer . . . , 7-mer. As far as purification was concerned, it was relatively easy because the resolution was greatly improved. As compared to the chemical method, the impurities were much less, high resolution was not required to complete the purification step, affording two advantages of waste gas reduction and extremely easy purification.

As shown in FIGS. 2, 3, 4, 5 and 6, 1 marked in the sequence represents the starting base 1 of the “DNA sequence to be synthesized.”

As shown in FIGS. 4 and 6, the sequences between −1 and 1 marked in the sequence, represent the enzymatic cleavage site, for example restriction endonuclease, nickase.

As shown in FIGS. 4 and 6, the number −1 to −10 were added to the front end of the first-order oligonucleotide, which were sequences provided for enzymatic recognition and cleavage, Sequences such as P5 were further included for primers to perform PCR reactions, allowing greater potentials for post-synthetic special applications, for example, amplifying the yields.

As shown in FIGS. 4, 5 and 6, the marked linker group or biotin is located at the 5 end of the complementary oligonucleotide, or it can also be placed at the 3′ end of the first-order oligonucleotide. In order to avoid the drawings from being too complicated, they are not shown in drawings, and can be understood by those skilled in the art.

The linker group LG mentioned in the present invention refers to a molecule that can be cleaved by chemical action, such as a disulfide bond, or a polypeptide that can be cleaved by protease cleavage, or a cleavable oligonucleotide that can be cleaved by nuclease.

The present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Those skilled in the art to which the present invention pertains can easily understand and realize the objects of the present invention and obtain the aforementioned results and advantages. The enzymes, solid-phase matrixes, nucleotides, oligonucleotide fragments, and processes and methods for producing them used in the present invention represent preferred embodiments, are exemplary in nature, and are not intended to limit the scope of the present invention. Those skilled in the art and the modifications or other uses that will occur when making or using this technology are all included in the spirit of the present invention and defined by the scope of rights.

Although this invention has been described and illustrated in sufficient detail to enable those skilled in the art to make and use it, various alternatives, modifications and improvements should be apparent without departing from the spirit and scope of the invention.

Those skilled in the art will readily appreciate that the present invention is well adapted to carry out the objects and obtain the objects and advantages mentioned, as well as those inherent therein. The processes and methods used to produce them are representative of preferred embodiments, are exemplary, and are not intended to limit the scope of the invention. Modifications therein and other uses will occur to those skilled in the art. These modifications are included within the spirit of the invention and defined by the scope of the claims.

1. A method for synthesizing DNA, which comprises:
(a) providing an oligonucleotide components library as a material for synthesizing DNA, wherein each oligonucleotide component is a short oligonucleotide chain whose new end and trailing end are OH groups;
(b) analyzing sequence information of a DNA to be synthesized to obtain an oligonucleotide components combination order;
(c) using the oligonucleotide components library to arrange an oligonucleotide component order according to the oligonucleotide components combination order;
(d) phosphorylating the new end of a first-order oligonucleotide;
(e) combining the first-order oligonucleotide with a complementary oligonucleotide of the first-order oligonucleotide to obtain a first-order double-stranded oligonucleotide;
(f) combining a second-order oligonucleotide in the oligonucleotide components combination order with a complementary oligonucleotide of the second-order oligonucleotide to obtain a second-order double-stranded oligonucleotide;
(g) ligating the phosphorylated new end of the first-order oligonucleotide with the trailing end of the second-order oligonucleotide to obtain a synthetic oligonucleotide having an OH group as the new end; and
(h) repeating the above steps (d) to (g) to sequentially elongate the synthetic oligonucleotide until the DNA sequence to be synthesized is completed.

2. The method of claim 1, wherein the short oligo library in step (a) is a basic component group or a custom-made component group.

3. The method of claim 1, wherein the order of steps (d) and step (e) is exchangeable.

4. The method of claim 1, wherein the first-order oligonucleotide sequence further comprises a nucleotide sequence capable of being recognized and cleaved by enzymes.

5. The method of claim 1, which further comprises the following steps after performing step (c) and before performing step (f):
binding the 3′ end of the first-order oligonucleotide sequence with a solid-phase matrix through a linker group, wherein the linker group is cleavable.

6. The method of claim 5, wherein the sequence of the linker group is provided with an oligonucleotide for enzymatic cleavage.

7. The method of claim 5, wherein the linker group is an oligonucleotide the sequence of which is complementary to the first-order oligonucleotide.

8. The method of claim 1, which further comprises the following step after step (h): eluting the complementary strand of the oligonucleotide of the synthetic oligonucleotide to obtain a synthetic single-stranded DNA.

9. The method of claim 8, wherein the complementary strand of the oligonucleotide sequence of the synthetic oligonucleotide is eluted by physical or chemical actions.

10. The method of claim 8, wherein the complementary strand of the oligonucleotide of the synthetic oligonucleotide is eluted by heating or changing pH values.

11. The method of claim 1, wherein the linker group is cleaved by heating, changing pH value or enzymatic decomposition.

12. The method of claim 1, wherein the new end of the first-order oligonucleotide in step (g) is ligated with the trailing end of the second-order oligonucleotide through an enzyme.

13. The method of claim 12, wherein the enzyme is a double-stranded DNA ligase.

14. The method of claim 1, wherein the length of the short oligonucleotide chain of the oligonucleotide sequence is n and n-1 bases, and n is an integer greater than or equal to 2.

15. The method of claim 14, wherein the n is 6-8.

16. The method of claim 14, wherein the n is 6.

17. The method of claim 1, wherein the length of the short oligonucleotide chain of the oligonucleotide is n and n-2 bases, and n is an integer greater than or equal to 3.

18. The method of claim 17, wherein n is 6-8.

19. The method of claim 1, wherein the length of the short oligonucleotide chain of the oligonucleotide sequence is n and n-3 bases, and n is an integer greater than or equal to 4.

20. The method of claim 19, wherein the n is 6-8.
